(19) United States
(12) Patent Application Publication
(10) Pub. No.: US 2014/0106010 A1
(43) Pub. Date: Apr. 17, 2014

(54) Blend of Green and Black Extracts for the Promotion of Health Benefits

(71) Applicant: Kemin Industries, Inc., Des Moines, IA (US)

(72) Inventors: Michael Ceddia, Ankeny, IA (US); Kelli Herrlinger, Johnston, IA (US); Diana Chirouzes, Des Moines, IA (US)

(73) Assignee: Kemin Industries, Inc., Des Moines, IA (US)

(21) Appl. No.: 14/054,193

(22) Filed: Oct. 15, 2013

Related U.S. Application Data
(60) Provisional application No. 61/764,017, filed on Feb. 13, 2013.
(60) Provisional application No. 61/713,896, filed on Oct. 15, 2012.

(51) Int. Cl. A61K 36/82

(52) U.S. Cl. CPC A61K 36/82
USPC 424/729

(57) ABSTRACT

A method of improving urological health in a human, including administration to the human of an effective amount of a water-extracted extract of both green and black tea.

This application claims priority to U.S. Patent Application Ser. No. 61/764,017, filed Feb. 13, 2013, and U.S. Patent Application Ser. No. 61/713,896, filed Oct. 15, 2012.

### BACKGROUND OF THE INVENTION

The prevalence of lower urinary tract symptoms (LUTS) increases during aging; about 25% of men in their 50's and 50% of men in their experience bothersome LUTS, which results in decreased quality of life. LUTS may be classified into voiding symptoms, storage symptoms, and post-micturition symptoms [Abrams P, Cardozo L, Griffiths D, Rosier P, Ulmsten U, Van Kerrebroeck P, Victor A, Wein A 2002 The Standardization of Terminology of Lower Urinary Tract Function Report from the Standardisation Sub-committee of the International Continence Society Neurourology and Urodynamics 21:167-178], and often result in poor patient-reported quality of life (QOL) (Wei J, Calhoun E, Jacobsen S: Urologic diseases in America project: benign prostatic hyperplasia. J Urol 2005; 173: 1256.). Voiding symptoms include trouble starting or continuing a urine stream, straining, dribbling, a weak or slow urine stream, and incomplete bladder emptying. Storage symptoms include may include passing urine often, especially at night or with sudden urge, and the major post-micturition symptom is post-micturition dribbling. Questionnaires such as the American Urological Association (AUA) Symptom Index or the International Prostate Symptom Score (IPSS) are frequently part of the diagnostic process to quantify the frequency and severity of LUTS. A maximum score on the AUA Symptom Index is 35 points, with 8 to 19 points indicating moderate symptoms and 20 to 35 points indicating severe symptoms.

LUTS are managed either through watchful waiting, drug therapy, or surgery (depending on etiology). Management is based on the extent of symptoms and impact on quality of life. Pharmacologic treatment for LUTS includes alpha-adrenergic antagonists (alpha-blockers (alfuzosin, doxazosin, tamsulosin, terazosin, sildosin)), 5-alpha-reductase inhibitors (5-ARIs (dutasteride, finasteride)), anticholinergic agents, and PDE-5 inhibitors (Auffenberg G, Helfan B, McVary K: Established medical therapy for benign prostatic hyperplasia. Urol Clin North Am 2009; 36: 443). Surgical therapy includes transurethral resection of the prostate (TURP), transurethral needle ablation (TUNA), transurethral microwave thermotherapy (TUMT), open prostatectomy and transurethral holmium laser ablation of the prostate (HoLAP) (AUA Practice Guidelines). Complementary approaches for treatment of LUTS include the use of dietary supplements and phytotherapeutic agents such as saw palmetto (Serenoa repens) and pygeum (Pygeum africanum); however, there have been contradictory results for these agents reported in the literature (Dvorkin L, Song K Y. Herbs for benign prostatic hyperplasia. 6. Ann Pharmacother. 2002 September; 36(9):1443-52; Tacklind J, MacDonald R, Rutks I, Wilt T J. Serenoa repens for benign prostatic hyperplasia. Cochrane Database Syst Rev. 2009 April 15; (2):CD001423).

Lower urinary tract symptoms (LUTS) are proposed to have a multi-factorial etiology. There are many possible causes of LUTS, with an enlarged prostate, also known as Benign prostatic hyperplasia (BPH), as the most common cause of LUTS. Additional causes include detrusor muscle weakness or over-activity, urinary tract infections, chronic prostatitis, urinary stones, or neurological dysfunction Inflammation, oxidative stress, and inhibition of apoptosis (resulting in a growth imbalance) have been considered as possible contributors to LUTS in the last decade. To remove confounding factors leading to LUTS etiology, clinical trials evaluating LUTS or LUTS treatments often enroll men with histologic BPH (confirmed) or LUTS suggestive of BPH (AUA Practice Guidelines Committee, authors. AUA guideline on management of benign prostatic hyperplasia (2003), chapter 1: diagnosis and treatment recommendations. J. Urol. 2003; 170(2 pt 1):530-547).

The prostate is a small gland in males that is part of the reproductive system and functions to help make semen. It is positioned below the bladder, just in front of the rectum, and surrounds part of the urethra. The three most common prostate issues are either classified as (Wei J, Calhoun E, Jacobsen S: Urologic diseases in America project: benign prostatic hyperplasia. J Urol 2005; 173: 1256) non-cancerous, namely inflammation (prostatitis), (McVary K: BPH: Epidemiology and Comorbidities. Am J Manag Care 12 2006; 5 Suppl: S 122) enlarged prostate, or (AUA Practice Guidelines Committee, authors. AUA guideline on management of benign prostatic hyperplasia (2003), chapter 1: diagnosis and treatment recommendations. J. Urol. 2003; 170(2 pt 1):530-547) cancerous, in the case of prostate cancer. As men age, the prostate tends to increase in size. An enlarged prostate may affect quality of life and cause problems in the urinary tract, bladder and kidney. Enlarged prostate affects about 50% of men in their 50s, with increasing prevalence of up to 90% in men in their 80s (Wei J, Calhoun E, Jacobsen S: Urologic diseases in America project: benign prostatic hyperplasia. J Urol 2005; 173: 1256; McVary K: BPH: Epidemiology and Comorbidities. Am J Manag Care 12 2006; 5 Suppl: S 122). An enlarged prostate may also result in LUTS. Causes and pathogenesis of BPH are largely unknown, although inflammation and apoptosis have been considered as possible causes in the last decade. Inflammatory infiltrates present in the prostates of BPH patients in the Medical Therapy of Prostate Symptoms (MTOPS) study were associated with an increased rate of disease progression.

In a large clinical trial, 1,145 men with moderate to severe LUTS undergoing pharmacologic therapy over 13 weeks could rate their condition as worse, or no, slight, moderate, or marked improvement, and associated a 3 point decrease in AUA symptom score as providing “slight improvement” and a 5.1 point decrease as “moderately improved” (Barry M J, Williford W O, Chang Y, et al. Benign prostatic hyperplasia specific health status measures in clinical research: How much change in the American Urological Association symptom index and the benign prostatic hyperplasia impact index is perceptible to patients? Journal of Urology 1995;154:1770-4). These men who considered their condition to be slightly improved over 13 weeks showed different minimal perceptible differences in the self-rated AUA Symptom Index, dependent upon baseline scores. Men with AUA Symptom Index baseline scores of 8 to 19 who felt their condition slightly improved exhibited a 2 point decrease and those who felt their condition was moderately improved exhibited a 4 point decrease, while men with baselines scores ≧20 who felt their condition slightly improved showed a 6 point decrease.

Alpha-blockers, which relax the muscles where the bladder narrows toward the urethra, and 5-α-reductase inhibitors, which shrink the size of the prostate gland, alone or in combination, have been shown to be effective drug treatments for reducing clinical progression of BPH and LUTS. Other common herbal supplements targeted for LUTS include beta-sitosterol, saw palmetto, pygeum, and nettles. If left untreated, LUTS may lead to a weak bladder, backflow of urine causing bladder or kidney infections, complete block in the flow of urine (also known as acute urinary retention), or kidney failure. Prostate cancer (PC) is the second leading cause of cancer death in men worldwide after lung cancer. PC begins as a localized lesion, and progresses to obtain invasive and metastatic potential, transitioning from an androgen-dependent to an androgen-independent state. Prostate intraepithelial neoplasia (PIN), a premalignant proliferation found within the prostate, commonly precedes PC by several years. Current evidence suggests men with LUTS are not at a greater risk of developing prostate cancer compared to asymptomatic men of the same age (Young, J. M., D. J. Muscatello, et al. (2000). “Are men with lower urinary tract symptoms at increased risk of prostate cancer? A systematic review and critique of the available evidence.” BJU International 85(9): 1037-1048.). In addition to LUTS with or without concurrent BPH, nutritional interventions have targeted decreasing PC disease risk as well as BPH, PIN, and PC progression, with tea emerging as a promising agent. Tea consumption has been associated with lower prostate cancer risk and the Mayo Clinic recommends drinking green tea to help prevent PC and other health problems [Prostate cancer prevention: What you can do. Mayo Clinic, 2009. (Accessed May 12, 2011, at http://www.mayoclinic.com/health/prostate-cancer-prevention/MC00027.)]. Non-prescription therapies for prostate problems are desired, with evidence that more than 25% of PC patients resort to alternative therapies including green tea and green tea extracts (Jacobson, J. S., and Chetty, A. P. (2001) Current oncology reports 3, 448-452). Green and black teas are a rich source of polyphenols, a powerful antioxidant. As a result of different processing techniques, the antioxidant profiles of green and black teas are slightly different. Green tea's freshly harvested leaves are steamed to prevent oxidation of polyphenols, while black tea leaves are purposefully withered, crushed, and fermented to allow oxidation of polyphenols. A typical cup of green tea brewed with 2 g dry tea leaves contains about one third (660 mg) water-extractable materials. Internal analysis at Kemin Human Nutrition and Health revealed a range of 109-176 mg total catechins and 180-243 mg total polyphenols per cup in commercially available brewed green teas. Green tea catechins include epigallocatechin-3-gallate (EGCG), epicatechin-3-gallate (ECG), epigallocatechin (EGC), and epicatechin (EC), with EGCG the most abundant of the catechins in green tea (about 100 to 150 mg per cup of brewed green tea). A similarly brewed cup of black tea contains 3-10% catechins, 2-6% theaflavins and >20% thearubigins in the water-extractable materials. The major theaflavin compounds include theaflavin (TF1), theaflavin-3-gallate (TF2A), theaflavin-3′-gallate (TF2B), and theaflavin-3,3′-digallate (TF3). Commercially available brewed black teas analyzed internally contained 4-8 mg theaflavins, 69-89 mg total catechins, and 167-214 total polyphenols per cup. Caffeine content in tea leaves is about 2-5% of water extractable materials. Both green and black teas have demonstrated anti-oxidant and anti-inflammatory activities; however, neither has been shown to have effectiveness in reducing lower urinary tract symptoms.

Green and black teas have been widely studied and large epidemiologic studies report links between tea consumption and reduced incidence of various diseases that have pathologies associated with mechanisms of oxidative stress and/or chronic inflammation such as obesity, diabetes mellitus, cardiovascular disease and urologic conditions such as BPH and PC (Hsu A, Bray T M, Ho E. Anti-inflammatory activity of soy and tea in prostate cancer prevention. Exp Biol Med (Maywood). 2010 June; 235(6):659-67; Bettuzzi et al., 2006). Inflammation has been proposed to be a contributing factor to bothersome LUTS, with studies showing that inflammatory cells and pro-inflammatory cytokines may also be involved in the proliferation of prostate tissues (Liao C H, Chung S D, Kuo H C. Serum C-reactive protein levels are associated with residual urgency symptoms in patients with benign prostatic hyperplasia after medical treatment. Urology. 2011 December; 78(6):1373-8). Data from the PC prevention trial demonstrated that elevated inflammatory biomarkers such as C-reactive protein and interleukin-6 might increase the risk of BPH (Schenk J M, Kristal A R, Neuhouser M L, Tangen C M, White E, Lin D W, Kratz M, Thompson I M. Biomarkers of systemic inflammation and risk of incident, symptomatic benign prostatic hyperplasia: results from the prostate cancer prevention trial. Am J. Epidemiol. 2010 March 1; 171(5):571-82). Basic science studies have demonstrated that green and black teas both have potent anti-inflammatory activities. For example, (−)-epigallocatechin-3-gallate (EGCG) present in both green and black teas has been shown to reduce expression of the p65 subunit of NFkB in vitro (Hastak K, Gupta S, Ahmad N, Agarwal M K, Agarwal M L, Mukhtar H. Role of p53 and NF-kappaB in epigallocatechin-3-gallate-induced apoptosis of LNCaP cells. Oncogene. 2003 Jul. 31; 22(31):4851-9) but no clinical studies have looked at green and black tea administration and a reduction in clinical LUTS.

Tea polyphenols have been recently detected in both animal and human prostate tissue (Henning, S. M., Aronson, W., Niu, Y., Conde, F., Lee, N. H., Seeram, N. P., Lee, R. P., Lu, J., Harris, D. M., Moro, A., Hong, J., Leung, P. S., Barnard, R. J., Ziaee, H. G., Csathy, G., Go, V. L. W., Wang, H., and Heber, D. (2006) Journal of Nutrition 136, 1839-1843). In C57BL/6 mice, 2 weeks of decaffeinated black tea extract (BTE) administration at 50 mg/g diet showed detectable levels of polyphenols and theaflavins in prostate tissue (ibid.). This experiment showed a 70% greater relative absorption of theaflavin into the prostate compared to Epigallocatechin-3-gallate (EGCG). Theaflavin and theaflavin-3-monogallate (from the BTE), and Epicatechin (EC) and Epigallocatechin (EGC) (from the GTE) were present primarily in their free forms in the prostate tissue. In a human study, twenty men with localized prostate cancer consumed one brewed green or black tea bag or soda control 5×/day for five days prior to prostate gland removal. Prostate concentrations of EGCG, EGC, and the sum of the four tea polyphenols were greater in both tea groups compared to control (Henning, S. M., Aronson, W., Niu, Y., Conde, F., Lee, N. H., Seeram, N. P., Lee, R. P., Lu, J., Harris, D. M., Moro, A., Hong, J., Leung, P. S., Barnard, R. J., Ziaee, H. G., Csathy, G., Go, V. L. W., Wang, H., and Heber, D. (2006) Journal of Nutrition 136, 1839-1843). Emerging evidence from animal and human studies shows that tea components are bioavailable in prostate tissue, where they may be able to exert protective effects.

### Green Tea Literature

The majority of studies exploring green tea and prostate function have centered on the compound EGCG and effects in PC. In vitro PC research uses androgen-dependent, LNCaP cell lines, or androgen independent, DU-145 and PC-3 cell lines. The transgenic adenocarcinoma of the mouse prostate (TRAMP) model is a widely used in vivo prostate model, which captures the progression of PC from PIN to an androgen-independent disease (Kaplan-Lefko, P. J., Chen, T. M., Ittmann, M. M., Barrios, R. J., Ayala, G. E., Huss, W. J., Maddison, L. A., Foster, B. A., and Greenberg, N. M. (2003) Prostate 55, 219-237). Green tea's proposed mechanisms of action in the prostate has been demonstrated through its ability to influence several pathways including apoptosis, inflammatory pathways, the HGF/c-MET pathway, insulin-like growth factor (IGF) axis, and the androgen receptor pathways.

In in vitro studies on PC cell lines, EGCG demonstrated dose-dependent promotion of apoptosis, or programmed cell death, and ultimately slowed the growth of PC. Evidence of changes in mitochondria, where the regulation of apoptosis occurs, include DNA fragmentation (Ahmad, N., Feyes, D. K., Nieminen, A. L., Agarwal, R., and Mukhtar, H. (1997) Journal of the National Cancer Institute 89, 1881-1886; Gupta, S., Ahmad, N., Nieminen, A. L., and Mukhtar, H. (2000) Toxicology and Applied Pharmacology 164, 82-90; Paschka, A. G., Butler, R., and Young, C. Y. F. (1998) Cancer Letters 130, 1-7) and cleavage of pro-caspases-3, -8, and -9 (Hastak, K., Gupta, S., Ahmad, N., Agarwal, M. K., Agarwal, M. L., and Mukhtar, H. (2003) Oncogene 22, 4851-4859) (found to be released in apoptosis). Promotion of apoptosis was also found through stabilization of the transcription factor p53 and a shift toward a pro-apoptotic ratio of BAX/Bcl-2. EGCG also has been shown to have effects on another transcription factor, nuclear factor-kappa B (NF-kB). Effects on NF-kB are important because of its proposed ability to regulate a variety of downstream targets of inflammation, cytokine production, and cell death. PC cells treated with 80 uM of EGCG, then stimulated by the inflammatory cytokine TNFα showed decreased NF-kB DNA binding activity and reduced expression of its p65 subunit (ibid.). In vivo experiments reinforce effects of EGCG on NF-kB. TRAMP mice fed 0.1% w/v green tea polyphenols in drinking water showed reduced expression of NF-kB and related proteins compared to control mice (Gupta et al., 2000; Siddiqui, I. A., Shukla, Y., Adhami, V. M., Sarfaraz, S., Asim, M., Hafeez, B. B., and Mukhtar, H. (2008) Pharmaceutical Research 25, 2135-2142). Another study showed treatment with green tea polyphenols (GTP) and EGCG reduced levels of the angiogenesis promoting vascular endothelial growth factor (VEGF) protein (Siddiqui, I. A., Zaman, N., Aziz, M. H., Reagan-Shaw, S. R., Sarfaraz, S., Adhami, V. M., Ahmad, N., Raisuddin, S., and Mukhtar, H. (2006) Carcinogenesis 27, 833-839). In vitro and in vivo studies show the ability of green tea components to influence proteins involved in apoptosis thus resulting in growth imbalance and an abnormal prostate environment,

The HGF/c-MET pathway, is considered an important regulator of pathways related to invasion and metastasis of many cancers. Hepatocyte growth factor (HGF) is the only known high-affinity ligand for the c-MET receptor tyrosine kinase. Once binding of HGF to c-MET occurs this leads to c-MET dimerization, autophosphorylation of its catalytic tyrosines, activation of the multi-functional docking site for adapter protein binding, and recruiting of signal transduction pathways such as RAS and phosphatidylinositol 3-kinase (PI3K) (Comoglio, P. M., Giordano, S., and Trusolino, L. (2008) Nature Reviews Drug Discovery 7, 504-516). In DU-145 PC cells exposed to HGF, addition of EGCG was able to prevent phosphorylation of tyrosines 1234/1235 in the kinase domain of the c-MET receptor without effecting dimerization, thus blocking cell scattering (Duhon, D., Bigelow, R. L. H., Coleman, D. T., Steffan, J. J., Yu, C., Langston, W., Kevil, C. G., and Cardelli, J. A. (2010) Molecular Carcinogenesis 49, 739-749). Studies have shown the ability of EGCG to have direct effects on the PI3K/Akt pathway and MAP Kinases (MAPKs), both involved in regulating cellular activities such as cell proliferation and cell survival. Identified MAPKs include extracellular signal-regulated kinases (ERK1 and ERK2), c-Jun N-terminal kinases (JNKs), p38 isoforms, ERK5, ERK3/4, ERK3 and ERK 7/8 (Albrecht, D. S., Clubbs, E. A., Ferruzzi, M., and Bomser, J. A. (2008) Chemico-Biological Interactions 171, 89-95). Not only was EGCG able to reduce phosphorylation of c-MET in vitro, but it also reduced phosphorylation of HGF-induced Akt and Erk (Duhon et al., 2010). In DU-145 and LNCaP cells in vitro, EGCG reduced PI3K levels and phosphorylation of Akt (Siddiqui, I. A., Adhami, V. M., Afaq, F., Ahmad, N., and Mukhtar, H. (2004) Journal of Cellular Biochemistry 91, 232-242). TRAMP mice fed 0.1% GTP or 0.06% EGCG in drinking water showed decreased phosphorylation of ERK1 and ERK2 by about 50% in the dorsolateral (Vayalil, P. K., and Katiyar, S. K. (2004) Prostate 59, 33-42) and ventral (Harper, C. E., Patel, B. B., Wang, J., Eltoum, I. A., and Lamartiniere, C. A. (2007) Prostate 67, 1576-1589) prostate. In the PI3K/Akt pathway, TRAMP mice fed green tea polyphenols showed lower PI3K levels and reduced phosphorylation of Akt (Adhami, V. M., Siddiqui, I. A., Ahmad, N., Gupta, S., and Mukhtar, H. (2004) Cancer Research 64, 8715-8722). The MAPK and PI3K/Akt signaling pathways may be regulated by the insulin like growth factor (IGF) axis, where green tea has shown inhibitory effects. Insulin-like growth factor binding proteins (IGFBPs) play a role in the transport of IGF's and modulation of their activity. Green tea polyphenols (0.1%) in the drinking water of TRAMP mice inhibited serum IGF-1, restored IGFBP-3 levels (Gupta, S., Hastak, K., Ahmad, N., Lewin, J. S., and Mukhtar, H. (2001) Proceedings of the National Academy of Sciences of the United States of America 98, 10350-10355), and altered their ratio (Adhami et al., 2004). In addition, the prostate showed decreased IGF-1 levels (Harper et al., 2007; Adhami et al., 2004) and IGF-1 receptor protein levels (Adhami et al., 2004). Evidence of green tea's protective effects within the HGF/c-MET pathway in vitro and in vivo indicate widespread implications in mechanisms that protect healthy cells and control abnormal cell growth,

One additional mechanism by which green tea exerts its protective effects on the prostate is through the androgen receptor. The androgen receptor is a nuclear receptor that remains in the cytoplasm in its inactive form bound to heat shock proteins. Upon binding with a ligand (such as testosterone or 5α-dihydrotestosterone) the androgen receptor can act as a DNA transcription factor for many genes such as prostate specific antigen (PSA). Evidence has emerged that EGCG plays a role as a direct antagonist of androgen action, resulting in decreased gene transcription and ultimately, the ability to repress androgen-induced PC cell growth (Siddiqui, I. A., Asim, M., Hafeez, B. B., Adhami, V. M., Tarapore, R. S., and Mukhtar, H. (2011) FASEB Journal 25, 1198-1207). EGCG has been shown to reduce gene expression and protein expression of androgen receptor in the androgen responsive LNCaP cell line (Ren, F., Zhang, S., Mitchell, S. H., Butler, R., and Young, C. Y. F. (2000) Oncogene 19, 1924-1932). An additional study showed that the in vivo administration of 0.1% GTP drinking water to athymic nude mice implanted with human PC CWR22Rv1 cells resulted in lower PSA levels compared to control animals, with in vitro evidence that EGCG may decrease the transcription and translation (i.e., both mRNA and protein levels) of PSA (Siddiqui et al., 2006). 5α-reductase, the enzyme that converts testosterone to DHT, is also involved in androgen signaling since testosterone and DHT are both ligands for the androgen receptor; however, DHT has a 4-5 fold higher affinity when compared to testosterone. Two different 5α-reductase isozymes exist, with the type 2 isozyme primarily expressed in prostate tissue (Andersson, S., and Russell, D. W. (1990) Proceedings of the National Academy of Sciences of the United States of America 87, 3640-3644), Although EGCG and ECG were found to inhibit the type I human isozyme (non-prostate tissue) in a cell free biochemical based assay, this was not observed in a whole cell based assay (Liao, S., and Hiipakka, R. A. (1995) Biochemical and Biophysical Research Communications 214, 833-838). There is evidence that green tea components are able to affect key control points in the androgen receptor, although most of this data has been demonstrated in vitro and in vivo. Research within human populations may further our knowledge of this important mechanism and have clinically relevant implications due to the frequent practice of monitoring PSA levels.

Animal and human studies have shown benefits on prostate outcomes with green tea consumption and early intake (Bettuzzi, S., Brausi, M., Rizzi, F., Castagnetti, G., Peracchia, G., and Corti, A. (2006) Cancer Research 66, 1234-1240; Kurahashi, N., Sasazuki, S., Iwasaki, M., and Inoue, M. (2008) American Journal of Epidemiology 167, 71-77; Jian, L., Xie, L. P., Lee, A. H., and Binns, C. W. (2004) International Journal of Cancer 108, 130-135; Adhami, V. M., Siddiqui, I. A., Sarfaraz, S., Khwaja, S. I., Hafeez, B. B., Ahmad, N., and Mukhtar, H. (2009) Clinical Cancer Research 15, 1947-1953). Consumption of 0.1% GTP in the drinking water of TRAMP mice delayed onset of PCand increased overall survival compared to water fed controls (Gupta et al., 2001). Administration before PCdevelopment and in the early stages of disease showed amplified protective effects (Adhami et al., 2009). In contrast, no benefit was observed when GTP supplementation began at 28 weeks, representing moderately differentiated carcinoma. In males with high-grade PIN (the main pre-malignant lesion of prostate cancer), although no changes were observed in PSA levels after one year of supplementation with 600 mg/day green tea catechins, tumor incidence was lower (Bettuzzi et al., 2006). A subset of these men who had coexistent high-grade PIN and BPH were evaluated for variation in LUTS; they had mild to moderate initial IPSS scores, with lower IPSS scores and improved quality of life related to cancer over three months. A 1.5-2 year follow-up period without supplementation revealed that GTP had long-lasting effects due to an 80% decrease in the progression of PIN to prostate cancer (Brausi et al 2008 Chemoprevention of Human Prostate Cancer by Green Tea Catechins: Two Years Later. A Follow-up Update European Urology).

### Black Tea Literature

Studies of black tea on the prostate are very limited. Black tea's mechanism of action in the prostate has mostly been explored in PC cell lines in vitro. Similar to green tea, black tea has shown effects on PI3K/Akt pathway, MAPKs, and androgen signaling. Black tea components have demonstrated additional effects on cell cycle progression and tumor suppression. Very few meaningful animal or human clinical studies on black tea and non-cancerous prostate conditions have been performed.

Apoptosis has been identified in PC cells treated wth theaflavins by shifts in the pro-apoptotic and anti-apoptotic proteins, caspase-3 and caspase-9 respectively (Kalra, N., Seth, K., Prasad, S., Singh, M., Pant, A. B., and Shukla, Y. (2007) Life Sciences 80, 2137-2146; Prasad, S., Kaur, J., Roy, P., Kalra, N., and Shukla, Y. (2007) Life Sciences 81, 1323-1331), dose-dependent increases in cytochrome C expression (Kalra et al., 2007), and dose-dependent loss of mitochondrial membrane potential (Prasad et al., 2007). Cell cycle arrest is known to be one of the underlying mechanisms of apoptosis. Cell cycle arrest was induced by theaflavins, namely through decreased expression of cyclin B and cdc25C proteins and increased p21 waf1/cip1 (Prasad et al., 2007). Increased expression of P53 (Kalra et al., 2007), an apoptosis intiator and tumor supressor, as well as changes in expression of its related Bax and Bcl-2 proteins (Kalra et al., 2007; Prasad et al., 2007), also supported increased apoptosis and reduced cell proliferation. Other observed in vitro changes induced by theaflavins included dose-dependent decreases in nuclear translocation of NF-kB and its p65 subunit, down-regulation of Erk1/2, decreases in phosphorylated p38 MAPK (Kalra et al., 2007), and reduced PI3K levels and phosphorylation of Akt (Adhami et al., 2004). Effects of theaflavins on androgen signaling include decreased androgen receptor protein (Siddiqui et al., 2004) and effects on CYP17. CYP17 is a crucial enzyme in the biosynthesis of androgens and catalyzes both 17α-hydroxylase and C_{17,20}-lyase. In vitro, theaflavins had more potent inhibitory activity against CYP17 (C_{17,20}-lyase) than catechins (Kimura, K. I., Itakura, Y., Goto, R., Tojima, M., Egawa, N., and Yoshihama, M. (2007) Bioscience, Biotechnology and Biochemistry 71, 2325-2328). This would favor the biosynthesis of glucocorticoids over sex hormones (Gilep, A. A., Sushko, T. A., and Usanov, S. A. (2011) Biochimica et Biophysica Acta—Proteins and Proteomics 1814, 200-209), which may have implications in slowing androgen dependent PC.

In athymic nude mice implanted with human PC CWR22Rv1 cells, water extract of black tea and theaflavins were able to inhibit growth of implanted prostate tumors, reduce serum PSA, reduce VEGF protein levels, and induce apoptosis (Siddiqui et al., 2006). Reactive oxygen species associated oxidative damage is well documented in PC and one in vivo study showed administration of BTE protected against losses in key antioxidant enzymes in the prostate. Administration of BTE restored levels of catalase, superoxide dismutase, lipid peroxidation, glutathione-s-transferase, and glutathione reductase in the prostate caused by testosterone-induced oxidative stress (Siddiqui, I. A., Raisuddin, S., and Shukla, Y. (2005) Cancer Letters 227, 125-132). Although few animal and human trials have studied the effects of black tea in non-cancerous prostate conditions, the finding that theaflavins and polyphenols have been detected in prostate tissue in vivo and in human clinical trials and data showing benefit in cascades known to affect prostate cancer led us to consider inclusion of black tea in a blended formula for benefit in a separate prostate-related condition, LUTS.

The majority of studies on tea and prostate health have been performed in patients with PC. While it is possible for individuals to have LUTS and PC at the same time, men experiencing LUTS and asymptomatic men of the same age have similar risk of developing prostate cancer (Young, J. M., D. J. Muscatello, et al. (2000). “Are men with lower urinary tract symptoms at increased risk of prostate cancer? A systematic review and critique of the available evidence.” BJU International 85(9): 1037-1048.). However, there are some common points between the two conditions. Symptoms experienced and risk profiles are similar. Prostate Specific Antigen (PSA), a protein released into the blood by the prostate, is a marker of prostate health often used for PCa screening, and in PCa and BPH management, PSA is typically present at very low levels in the blood, although levels increase as the prostate enlarges. The PSA level is one of the most useful tests for early detection of prostate cancer (Medicine, J. H. (2011) Prostate Cancer. Remedy Health Media, LLC). The National Cancer Institute classifies a PSA level of 0-2.5 ng/dL as low, 2.6-10 ng/dL as a slightly to moderately elevated level, 10-19.9 ng/dL as moderately elevated, and ≧20 ng/dL as significantly elevated. A progressive increase in PSA obtained through follow-up measurements may indicate cancerous growth or in individuals with BPH a high PSA level has been predictive of complications such as acute urinary retention and a possible need for BPH-related surgery (Roehrborn, C. G. (2008) BJU International, Supplement 101, 17-21). Although minimal human research has been conducted on the effect of tea in non-cancerous prostate conditions, studies in PC patients may provide some insight and serve as a guide for future research in other prostate conditions.

We are not aware of any experimental studies examining the combination of green tea and black tea on prostate health. Since inflammation, oxidative stress, and apoptosis have been implicated as contributors to LUTS, compounds targeting these intracellular signaling pathways show promise. Green tea and its active components have been shown to promote apoptosis and repress prostate tumor growth through DNA damage protection, stabilization of the transcription factor p53, and reduced expression of NF-kB and related proteins. Green tea influences cell proliferation and cell survival through decreased phosphorylation of proteins in the HGF/c-MET pathway, downstream MAPKs, and PI3K/Akt. Additional actions of green tea include inhibition of IGF-1 and decreased expression of androgen receptor. Studies of black tea on the prostate are just emerging; however, black tea and its components show effective cell protection similar to green tea components. This is supported by studies that have shown that black tea influences the cell cycle arrest proteins cyclin B, cdc25C, and p21 waf1/cip1 along with protection of antioxidant enzymes in the prostate in the presence of oxidative stress. Together, active antioxidant compounds in green and black teas may have complementary effects for the prostate under conditions of oxidative stress or inflammation.

### SUMMARY OF THE INVENTION

The present invention consists of a method for improving urological health, alleviating LUTS and BPH symptoms (e.g., post void residual volume, AUA Symptom Scores, nocturia, weak stream, urinary frequency, urinary intermittency, incomplete emptying, inability to perform activities of daily living, and sexual desire and function), alleviating physiological risk factors (e.g., reducing inflammation [CRP], oxidative stress, blood pressure), by the administration of a therapeutically effective amount of a combination of water-extracted extracts of green and black tea. The green tea and black tea extract (GBTE) blends of the present invention are prefereably standardized to contain a minimum of 40% polyphenols, a minimum of 20% catechins and theaflavins (combined), and between 7% and 14% epigallocatechin-3-gallate (EGCG) and, in a preferred embodiment, consists of a compositional fingerprint similar to that shown in FIG. 4, wherein catechins and theaflavins in the product are identified.

An object of the present invention is an antioxidant composition comprising a blend of an extract of green tea and an extract of black tea that has greater antioxidant activity than the same amount of either extract alone.

Another object of the present invention is an antioxidant composition, wherein the blend is between 30% and 70% green tea extract and between 70% and 30% black tea extract.

A further object of the present invention is an antioxidant wherein the extracts are water extracts.

An object of the present invention is an antioxidant composition comprising a blend of an extract of green tea and an extract of black tea that has greater antioxidant activity than the sum of the antioxidant activity of the same amounts of the green tea and black tea extracts.

Another object of the present invention is a method of improving urological health in a human, comprising administering an effective amount of an extract of green tea and an extract of black tea that has greater antioxidant activity than the same amount of either extract alone.

A further object of the present invention is a method of reducing the AUA Symptom Score in a human, comprising administering an effective amount of an extract of green tea and an extract of black tea that has greater antioxidant activity than the same amount of either extract alone.

An object of the present invention is a method of reducing inflammation in a human, comprising administering an effective amount of an extract of green tea and an extract of black tea that has greater antioxidant activity than the same amount of either extract alone.

Another object of the present invention is a method of ameliorating lower urinary tract symptoms in a human, comprising administering an effective amount of an extract of green tea and an extract of black tea that has greater antioxidant activity than the same amount of either extract alone.

A further object of the present invention is a method of improving sexual desire in a human, comprising administering an effective amount of an extract of green tea and an extract of black tea that has greater antioxidant activity than the same amount of either extract alone.

An object of the present invention is a method of improving quality of life through improved physical functioning, including but not limited to activities of daily living, in a human, comprising administering an effective amount of an extract of green tea and an extract of black tea that has greater antioxidant activity than the same amount of either extract alone.

Another object of the present invention is a method of ameliorating benign prostatic hyperplasia (BPH) in a human, comprising administering an effective amount of an extract of green tea and an extract of black tea that has greater antioxidant activity than the same amount of either extract alone.

A further object of the present invention is a method of ameliorating nocturia in a human, comprising administering an effective amount of an extract of green tea and an extract of black tea that has greater antioxidant activity than the same amount of either extract alone.

An object of the present invention is a method of ameliorating weak stream in a human, comprising administering an effective amount of an extract of green tea and an extract of black tea that has greater antioxidant activity than the same amount of either extract alone.

Another object of the present invention is a method of ameliorating urinary frequency in a human, comprising administering an effective amount of an extract of green tea and an extract of black tea that has greater antioxidant activity than the same amount of either extract alone.

A further object of the present invention is a method of ameliorating urinary intermittency in a human, comprising administering an effective amount of an extract of green tea and an extract of black tea that has greater antioxidant activity than the same amount of either extract alone.

An object of the present invention is a method of ameliorating incomplete emptying in a human, comprising administering an effective amount of an extract of green tea and an extract of black tea that has greater antioxidant activity than the same amount of either extract alone.

Another object of the present invention is a method of ameliorating oxidative stress in a human, comprising administering an effective amount of an extract of green tea and an extract of black tea that has greater antioxidant activity than the same amount of either extract alone.

A further object of the present invention is a method of reducing blood pressure in a human, comprising administering an effective amount of an extract of green tea and an extract of black tea that has greater antioxidant activity than the same amount of either extract alone.

An object of the present invention is a method of reducing fasting blood glucose in a human, comprising administering an effective amount of an extract of green tea and an extract of black tea that has greater antioxidant activity than the same amount of either extract alone.

### BRIEF DESCRIPTION OF THE FIGURES

FIG. 1 is a chart of the AUA symptom score for each treatment group.

FIG. 2 is a chart of the C-reactive protein assay for each treatment group showing percent change from baseline.

FIG. 3 is a chart of the post void residual volume for each treatment group showing percent change from baseline.

FIG. 4 is a characteristic HPLC-UV chromatogram of the green and black tea blend of the present invention.

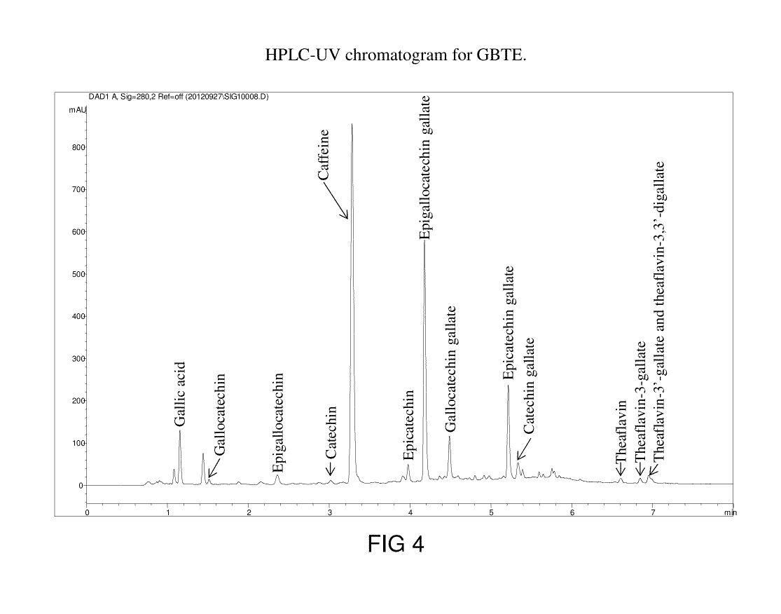

FIG. 5 is a chart of the ferric reducing antioxidant power (FRAP) assay of the green and black tea blend of the present invention.

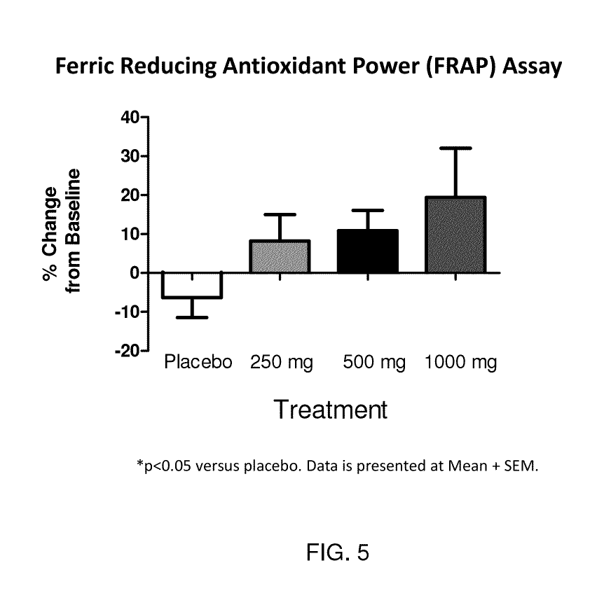

FIG. 6 is a chart of the effect of a black tea extract, a green tea extract, or a black and green tea extract blend employing a UV/VIS spectrophotometric method based on reduction of a 2,2-Diphenyl-1-picryl-hydrazyl (DPPH) free radical.

FIG. 7 is a chart of the effect of three different doses of a blend of black tea and green tea extracts on blood glucose levels.

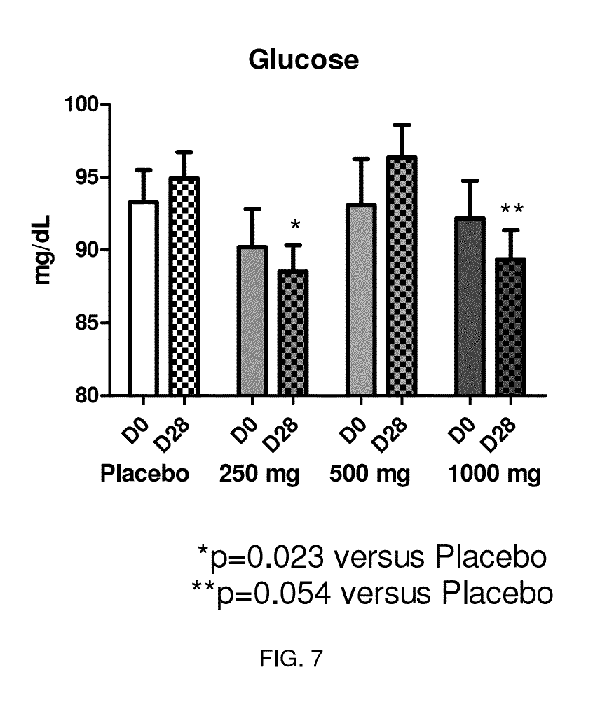

### DESCRIPTION OF THE INVENTION

The terms “administration of” or “administering a” compound should be understood to mean providing a compound of the invention to the individual in need of treatment in a form that can be introduced into that individual's body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, the term “extract” refers to a product prepared by extraction. The extract may be in the form of a solution in a solvent, or the extract may be a concentrate or essence which is free of, or substantially free of solvent. The term extract may be a single extract obtained from a particular extraction step or series of extraction steps or the extract also may be a combination of extracts obtained from separate extraction steps. For example, extract “a” may be obtained by extracting cranberry with alcohol in water, while extract “b” may be obtained by super critical carbon dioxide extraction of cranberry. Extracts a and b may then be combined to form extract “c”. Such combined extracts are thus also encompassed by the term “extract”.

As used herein, the term “fraction” means the extract comprising a specific group of chemical compounds characterized by certain physical, chemical properties or physical or chemical properties.

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

By “pharmaceutically acceptable” it is meant the carrier, diluent or excipient must be compatible with the other ingredients of the formulation and not deleterious to the recipient thereof.

The term “synergistic” is well understood in the art and refers to two or more components working together so that the total effect is greater than the sum of the components.

The term “treating” is well understood in the art and refers to curing as well as ameliorating at least one symptom of any condition or disorder.

The term “prophylactic or therapeutic” treatment is well understood in the art and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds.

The compounds of this invention may be administered to patients (humans and animals, including companion animals, such as dogs, cats and horses) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

A suitable dosage level of GBTE of the present invention is about 25 to 2000 mg per day, which may be given as a single dose or divided into two or three doses per day. Preferably, the dosage range will be about 50 mg to 2000 mg per subject per day; more preferably about 250 to 500 or 1000 to 1500 mg per subject per day.

A suitable dosage level of the blend of green and black tree extract (GBTE) is about 0.01 mg to 5.0 mg/kg per day, in particular about 0.1 to about 2.5 mg/kg, such as from about 0.5 to about 10 mg/kg per day. The dosage range will generally be about 50 to 2000 mg per subject per day, which may be administered in single or multiple doses. Preferably, the dosage range will be about 100 mg to 2000 mg per subject per day; and more preferably about 250 mg to 1000 mg per subject per day. Specific dosages of the compounds of the present invention for administration include 50 mg, 100 mg, 250 mg, 500 mg, 1000 mg, and 2000 mg. Compositions of the present invention may be provided in a formulation comprising about 25 mg to 2000 mg active ingredient; more preferably comprising about 50 mg to 1000 mg active ingredient; or 100 mg to 500 mg active ingredient; or 250 mg to 1000 mg active ingredient.

The GBTE is a combination of water extracts of green tea and water extracts of black tea. Preferably, the GBTE is between about 30% and 70% of a water extract of green tea and between about 70% and about 30% of a water extract of black tea, more preferably between about 40% and 60% of a water extract of green tea and between about 60% and about 40% of a water extract of black tea, such as a 50/50 blend of water extracts of green and black tea. The GBTE is preferably standardized to contain a minimum amount of polyphenols, a minimum amount of catechins, a minimum amount of theaflavins, or/and a minimum amount of epigallocatenchin-3-gallate (EGCG). The range of polyphenols is preferably between about 10% by weight of the GBTE and about 90% by weight of the GBTE, more preferably between 10% and 70%, and even more preferably between 30% and 60%, such as 40% or greater. The range of catechins and theaflavins is preferably between about 5% by weight of the GBTE and about 60% by weight of the GBTE, more preferably between 10% and 50%, and even more preferably between 20% and 40%, such as 20% or greater. The range of EGCG is preferably between about 1% by weight of the GBTE and about 30% by weight of the GBTE, more preferably between 3% and 20%, and even more preferably between 7% and 14%.

### Example 1

The aim of the conducted clinical study was to assess a novel blend of green and black tea extract, GBTE, in a human clinical study of men with moderate to severe lower urinary tract symptoms (LUTS). GBTE is a unique water-extracted tea ingredient that also has potent antioxidant activity. In this study, GBTE was administered to male subjects with LUTS in a randomized, double-blind, placebo controlled study in order to test the efficacy of two doses of the study agent to reduce LUTS over 12 weeks. To our knowledge, this was the first study exploring the effects of a blended green and black tea extract in men with LUTS.

### Patients and Methods

### Study Design

This was a prospectively designed IRB approved (Quorum Institutional Review Board, Seattle, Wash.) human clinical trial performed in accordance with Good Clinical Practices. Eligibility criteria included men who were 30-70 years of age, had moderate to severe LUTS at screening as assessed with an AUA symptom Score (AUA)≧8 and ≦24, and were willing to provide written informed consent. Men were excluded based on the exclusion criteria in Table 1.

TABLE 1
Exclusion Criteria

Current use of any medical therapy for BPH including 5-alpha reductase inhibitors
or alpha blockers (washout period was defined)
Past surgical procedure for BPH symptoms
Taking an oral alpha agonist, tricyclic antidepressant and anticholinergic or
cholinergic medication within 4 weeks of the screening visit
Any estrogen or androgen use within the past 3 months
Alanine transaminase (ALT), aspartate transferase (AST) or gamma-glutamyl
transpeptidase (GCT) value greater than 3 times the upper limit of normal
Prostate specific antigen > 10 ng/ml
History or current evidence of carcinoma of the prostate, pelvic radiation, urethral
stricture or prior surgery for bladder neck obstruction
Active urinary tract infection
Greater than 2 urinary tract infections within the past year
Cystoscopy or prostate biopsy within past 4 weeks or any prostate biopsy planned
during the 12-week study period
History of acute or chronic bacterial prostatitis within the past 6 months
Known severe bleeding disorder
Cancer not considered cured (except basal cell or squamous cell carcinoma of the
skin)
Hypersensitivity or known allergy to green tea or black tea
Hypersensitivity or known allergy to caffeine
Current use of any dietary supplement containing green or black tea
Current use of any dietary supplement for LUTS including saw palmetto, B-
sitosterol, pygeum (washout period was defined)
Current use of any dietary supplements that may affect anti-oxidant status including:

Vitamin E >400 IU, Vitamin C >500 mg, CoQ-10, lycopene, resveratrol,
pycnogenol, Any formulation similarly named “Antioxidant formula”, any
other dietary supplement in the opinion of the investigator may affect
antioxidant levels (washout period was defined).

Current intake of more than 1 cup of green or black tea per day
Current intake of more than 2 cups of coffee or other caffeinated beverage (includes
energy drinks, soda, etc.) per day
Unable to follow protocol directions due to organic brain or psychiatric disease
Planned cataract surgery during the course of the study
Recent history (greater than two years) of alcoholism or unlikely to refrain from
excessive alcohol consumption during the study period defined as >2 alcoholic
drinks/day
Current smoking or smoking within the past 3 months
Use of any recreational drugs or a history of drug addiction

Patients who met eligibility criteria were randomized to one of three study arms, 500 mg GBTE, 1000 mg GBTE or placebo (baseline demographics are shown in Table 2). Evaluable subjects were defined prospectively as those subjects completing the trial with a compliance of ≧80%.

TABLE 2
Demographics

| | Arm 1: | Arm 2: | Arm 3: | p- |
|---|---|---|---|---|
| Characteristic | 500 mg | 1000 mg | Placebo | value |
| N | 15 | 13 | 12 | n/a |
| Mean age, years | 56.77 (2.83) | 55.94 (1.71) | 57.68 (2.11) | 0.925 |
| (SD) | | | | |
| BMI | 27.96 (2.57) | 27.79 (1.77) | 30.59 (2.39) | 0.135 |
| AUA Symptom | 19.33 (1.02) | 17.85 (1.04) | 18.42 (1.35) | 0.818 |
| Score | | | | |

Mean (SEM)

The primary outcome evaluated was the score on the American Urological Association Symptom Index, which quantifies lower urinary tract symptom frequency and severity. Secondary measures included C-reactive protein (CRP), measurement of post void residual (PVR), and scores on the Short-Form 36 (SF-36) quality of life and International Erectile Function Index (IIEF) quantitative questionnaires. All measures were assessed at all time points, baseline (BL), mid-point (week [wk] 6), and endpoint (wk 12).

### American Urologic Association Symptom Index AUA-SI

The AUA-Symptom index is a self-reported questionnaire that was developed and validated in 1992 and quantifies symptom frequency and severity (Ko Y H, Chae J Y, Jeong S M, Kang J I, Ahn H J, Kim H W, Kang S G, Jang H A, Cheon J, Kim J J, Lee J G. Clinical Implications of Residual Urine in Korean Benign Prostatic Hyperplasia (BPH) Patients: A Prognostic Factor for BPH-Related Clinical Events. Int Neurourol J. 2010 December; 14(4):238-44). It is short, reliable, and comprised of 7 questions for the evaluation of urinary stream, frequency, intermittence, emptying, urgency, strain, and nocturia.

### Post-Void Residual Volume (PVR)

The measurement tool for post-void residual is a bladder scan conducted using ultrasonography. The bladder scan is a quick and non-invasive method of assessing bladder volume to determine the amount of urine retention or post-void residual urine volume (AUA Practice Guidelines).

### Short-Form 36 (SF-36)

The SF-36 is a multi-purpose, short-form health survey with 36 questions. It yields two summary measures of physical and mental health, which are further broken down into an 8-subscale profile for physical functioning, role of physical health, bodily pain, general health, vitality, social functioning, role of emotional health, and mental health (McHorney C A, Ware J E Jr, Lu J F, Sherbourne C D. The MOS 36-item Short-Form Health Survey (SF-36): III. Tests of data quality, scaling assumptions, and reliability across diverse patient groups. Med Care. 1994 January; 32(1):40-6).

### The International Index of Erectile Function (IIEF)

The IIEF was designed to assess erectile function in men. It is a well-validated self-reported questionnaire with 5 subsections for erectile function, orgasmic function, sexual desire, intercourse satisfaction, and overall satisfaction (Rosen R C, Cappelleri J C, Gendrano N 3rd. The International Index of Erectile Function (IIEF): a state-of-the-science review. Int J Impot Res. 2002 August; 14(4):226-44. Review).

### Study Agent

The study agent was an encapsulated dietary supplement for a total dose of 0 mg (placebo), 500 mg GBTE or 1000 mg GBTE per day. The study agent was formulated under Good Manufacturing Practices, produced, encapsulated and packaged in 120 capsule quantities, light resistant plastic bottles. The product lots were tested for toxins including heavy metals, pesticides and excipients. The placebo agent contained an inert substance and was aesthetically matched to the study agent. Stability of the capsules was confirmed throughout the study period.

### Statistical Analyses

Statistics are presented as mean±SEM for normally distributed variables (e.g., age). Outcome scores were evaluated using repeated measures analysis of variance performed by SAS Proc Mixed. The main effects of treatment and time were evaluated along with the interaction between these factors. Contrasts of interest were constructed and individually evaluated. Typically change scores, that is, the difference between a given post baseline (BL) score and the value of that variable at BL, served as the dependent variable in any given analysis of variance model. Although descriptive statistics were computed for the raw values of BL and post treatment outcome raw scores, the Least Squares Means were used to summarize findings for change scores that were evaluated by analysis of variance. All tests were two tailed and statistical significance was defined at p≦0.05. Change scores from BL at the post treatment time points (wk 6 and wk 12) were analyzed for the variables AUA, CRP, PVR, IIEF, and SF-36 (wk 12 only).

### Results

### Subjects

Fifty-nine subjects were screened, 46 of which were entered into the study and 40 completed the study and were defined as evaluable upon study completion. Treatment groups were well balanced for demographics and clinical characteristics (Table 2). Mean age was 56.77 (1.33), (Mean [SEM]). AUA symptom score was not different between subjects among the three groups at baseline, p=0.818 (Table 2).

### AUA Symptom Score

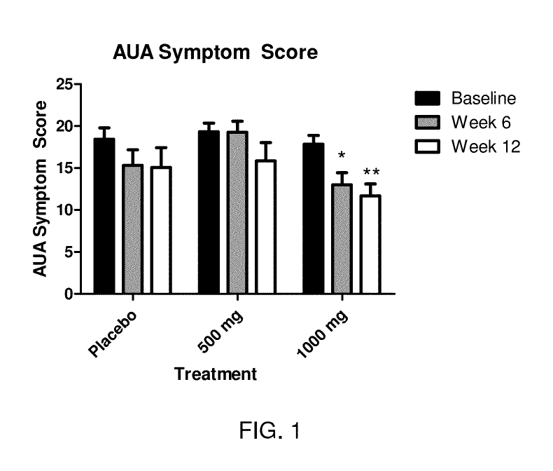

The mean value of the AUA symptom score decreased 34.5% over 12 weeks from BL in the 1000 mg GBTE group, with scores of 17.85±1.04, 13.00±1.45, and 11.69±1.42, at BL, wk 6 (p=0.0349 vs. BL), and wk 12 (p=0.0083 vs. BL), respectively (FIG. 1). Scores in the 500 mg GBTE group were 19.33±1.02, 19.27±1.31, 15.87±2.16 (BL, wk 6, and wk 12, respectively) and the placebo group scores were 18.42±1.35, 15.33±1.85, and 15.08±2.36 (BL, wk 6 and wk 12, respectively). There were no significant differences in AUA score changes for either the 500 mg GBTE or placebo group at any timepoints in comparison to BL.

A further breakdown of AUA Score to determine which of the 7 questions that compose the score were primarily responsible for the observed overall AUA differences found that questions 1,3,5, and 7 were major contributing factors to the observed decrease in the 1000 mg GBTE treatment group. Each question is scored on a scale of 1-5 with 1 indicating the least symptoms while a 5 indicates greatest symptom severity, the 7 questions therefore can combine to total 35 points, with a decrease in score indicative of symptom improvement.

Question 1 pertained to incomplete emptying and asked “Over the past month, how often have you had a sensation of not emptying your bladder completely after you finished urinating?” In 1000 mg GBTE group, the change in score reported at wk 12 versus BL was statistically significant (2.85±0.25[BL], 1.83±0.27[week 12], p=0.0255), while the placebo and 500 mg GBTE were not significantly changed at wk 12 in comparison to BL.

Question 3 evaluated urinary stream intermittency by asking “Over the past month, how often have you found that you stopped and started again several times after you urinated?” In 1000 mg GBTE, the change in score reported at wk 6 compared to BLwas significantly decreased (2.31±0.37 [BL], 1.46±0.31 [wk 6], p=0.0410), while the placebo and 500 mg GBTE were not significantly changed at wk 6 versus BL.

Question 5 pertained to a weak urinary stream, asking “Over the past month, how often have you had a weak stream?’ The change from BL in the 1000 mg GBTE group (−1.05±0.41) versus placebo (−0.09±0.42) at wk 12 was significant (p=0.0559). In addition, for the 1000 mg GBTE group, wk 12 versus BL was statistically significant (2.54±0.35 [BL], 1.64±0.36 [wk 12], p=0.0141).

Finally, Question 7 evaluated nocturia, probing “Over the past month, how many times did you get up to urinate from the time you went to bed until the time you got up in the morning?” Change from BL comparison for all groups found the model was statistically significant (p=0.0415). In addition, 1000 mg GBTE treatment found placebo was statistically different than wk 6 (2.69±0.29 [BL], 1.54±0.18 [wk 6], p=0.002), and wk 12 (1.67±0.28 [wk 12], p=0.0046).

### Secondary Measurements

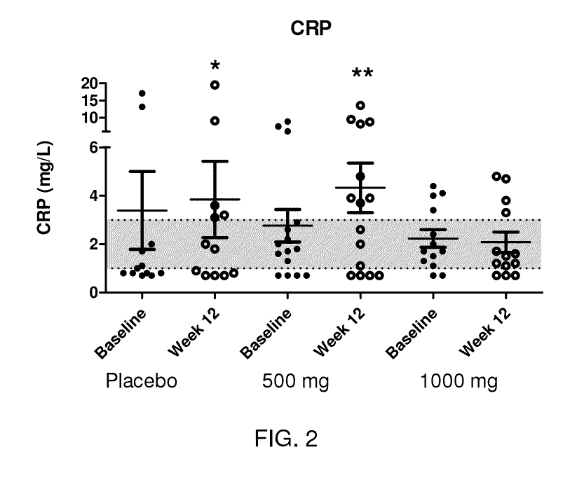

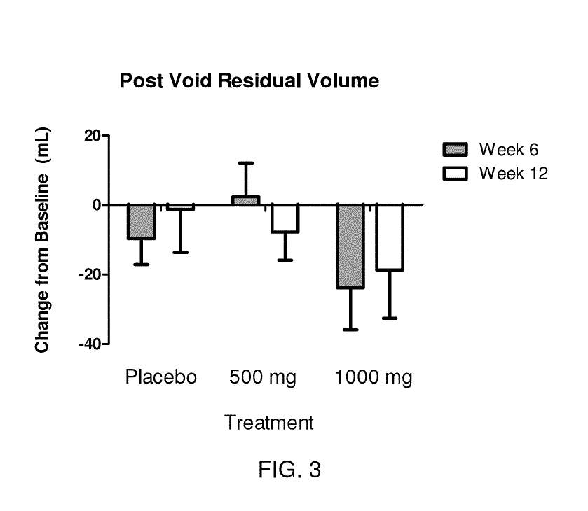

C-reactive protein (CRP) significantly increased in the placebo group (BL, 3.39±1.61 versus wk 12, 3.84±1.58 [p=0.0112]) and 500 mg GBTE (BL, 2.76±0.67 versus wk 12, 4.33±1.02 [p=0.0025]) versus the 1000 mg group which remained unchanged from BL to wk 12 (BL, 2.23±0.36 versus wk 12, 2.08±0.42) (FIG. 2). Post-void residual (PVR) demonstrated that there was a significant reduction in PVR in the 1000 mg GBTE group at wk 6 (41.55 mL±10.10) versus BL (65.39 mL±13.22, p=0.0338) (FIG. 3).

### IIEF

Breakdown of the IIEF score into subsections demonstrated changes in the question(s) evaluating Sexual Desire. The overall model had a significant group effect (p=0.0047). 1000 mg GBTE resulted in an increase in the sexual desire question(s) versus BL at wk6 (p=0.0413) and at wk 12 (p=0.0147), with no change identified in the placebo group.

### SF36

Subsectioning of the SF36 score showed that the 10 questions designated for the evaluation of physical function had an overall group difference (p=0.0509). Evaluation of the change from BL for the 1000 mg GBTE group versus placebo, showed increases in feeling of physical function at wk12 (p=0.0079).

### Blood Pressure

At wk 6, diastolic blood pressure statistically decreased in the 500 mg GBTE group in comparison to placebo (p=0.02), while diastolic blood pressure in the 1000 mg GBTE group decreased in comparison to placebo at week 6 (p=0.0027).

Change in systolic blood pressure from BL demonstrated decreases in the 500 mg GBTE group in comparison to placebo(p=0.09). At 1000 mg GBTE the change in systolic blood pressure from BL at wk 6 was found to decrease (p=0.03), and the change from BL at wk 12 showed a trend at p=0.08.

### Discussion

Epidemiological studies have showed the potential benefit of green and black tea in reducing the incidence of a number of conditions such as cardiovascular disease, metabolic disorder and cancer (including prostate cancer) (Bettuzzi et al., 2006). This study was the first to analyze the effect of administration of a blended, water-extracted green and black tea extract in reducing lower urinary tract symptoms (LUTS) in men with moderate or severe LUTS. The current study demonstrated that supplementation with 1000 mg of a blend of green and black tea extracts resulted in a 34.5% significant reduction in American Urological Association (AUA) symptom scores, in comparison to no change observed in the 500 mg GBTE or placebo administration over 12 weeks. This data is similar to a number of studies testing pharmacological agents such as the alpha-blocker tamsulosin (Flannery M T, Ramsdell J, Ranhosky A, Davidai G, Ruoff G. Efficacy and safety of tamsulosin for benign prostatic hyperplasia: clinical experience in the primary care setting. Curr Med Res Opin. 2006 April; 22(4):721-30) and better than some studies testing dietary supplement agents such as saw palmetto (6, MacDonald R, Tacklind J W, Rutks I, Wilt T J. Serenoa repens monotherapy for benign prostatic hyperplasia (BPH): an updated Cochrane systematic review. BJU Int. 2012 June; 109(12): 1756-61). Patients undergoing therapy who perceived their condition to be slightly improved over 13 weeks reported different perceptible differences in the self-rated AUA Symptom Index which was dependent upon BL scores. Men with AUA Symptom Index B L scores of 8 to 19 who felt their condition slightly improved showed a 2 point decrease, while men with a BL score greater than 20 who felt their condition slightly improved showed a 6 point decrease. In addition, evaluation of changes over 13 weeks of men with BL AUA symptom scores from 0 to 35, reported a 3 point decrease in AUA symptom score as providing “slight improvement” and a 5.1 point decrease as “moderately improved.” (Barry M J, Fowler F J Jr, O'Leary M P, Bruskewitz R C, Holtgrewe H L, Mebust W K, Cockett A T. The American Urological Association symptom index for benign prostatic hyperplasia. The Measurement Committee of the American Urological Association. J. Urol. 1992 November; 148(5):1549-57). Most studies interpret changes in the population with LUTS in comparison to baseline. In addition to showing statistically significant improvements in the AUA symptom score versus baseline in the 1000 mg GBTE group at 6 and 12 weeks, this trial also demonstrates clinically relevant improvements over the placebo at 12 weeks. Over 12 weeks, the subtraction of the placebo effect (3.34 point decrease in AUA-SI from baseline) from the effect of 1000 mg GBTE (6.16 point decrease in AUA-SI from baseline) yields a 2.82 point difference. Given that this population's initial AUA symptom score was 18.53±0.64, although there was no statistically significant difference over placebo, this 2.82 point decrease confirms a clinically relevant improvement versus placebo over 12 weeks. The data in the current study therefore indicates benefit to individuals with both moderate and severe LUTS following consumption of GBTE.

The results of this study demonstrate the ability of 1000 mg of GBTE to prevent the increases in inflammation that occurred in the placebo and 500 mg GBTE LUTS population. The biomarker used for the evaluation of inflammation in this study was C-reactive protein (CRP). Literature shows that CRP levels increase with increasing symptom severity in subjects with LUTS. In the current study, all participating individuals agreed to discontinue any medications or supplements for the treatment of their symptoms to remove the possibility of any confounding factors within the study design. It is therefore possible that individuals, who were randomly assigned to the placebo group may have experienced a worsening of their symptoms and thus an increase in their associated CRP levels. This suggests that administration of 1000 mg of GBTE supplement, may be acting to reduce LUTS through an anti-inflammatory mechanism of action, as it has been suggested that inflammation may be a predictor of LUTS (Liao C H, Chung S D, Kuo H C. Serum C-reactive protein levels are associated with residual urgency symptoms in patients with benign prostatic hyperplasia after medical treatment. Urology. 2011 December; 78(6):1373-8; Menschikowski M, Hagelgans A, Fuessel S, Mareninova O A, Neumeister V, Wirth M P, Siegert G. Serum levels of secreted group IIA phospholipase A(2) in benign prostatic hyperplasia and prostate cancer: a biomarker for inflammation or neoplasia? Inflammation. 2012 June; 35(3):1113-8).

The current study also demonstrated that there was a significant reduction in post-void residual volume at wk 6 versus BL in the 1000 mg GBTE group, while no observed decreases in residual volume remaining in the bladder at wk 6 versus BL in the 500 mg GBTE and placebo groups. Recent studies have shown that increased PVR at BL may be related to BPH clinical events including increased AUA (or International Prostate Symptom Score) Scores (Ko Y H, Chae J Y, Jeong S M, Kang J I, Ahn H J, Kim H W, Kang S G, Jang H A, Cheon J, Kim J J, Lee J G. Clinical Implications of Residual Urine in Korean Benign Prostatic Hyperplasia (BPH) Patients: A Prognostic Factor for BPH-Related Clinical Events. Int Neurourol J. 2010 December; 14(4):238-44). In addition, residual urine remaining in the bladder can result in incontinence, increased frequency of urination, urinary tract infections, nocturia, and in extreme neglected cases chronic renal failure.

While 500 mg GBTE was not effective over 12 weeks at significantly reducing post-void residual volume or AUA score, by week 12 decreases in the desired direction were occurring. For example, from Week 6 to week 12, there was no difference in the percent of subjects who achieved a clinically significant improvement in the placebo or 1000 mg GBTE group. However, an additional 20% of subjects consuming 500 mg/day of GBTE achieved a clinically significant improvement in AUA symptoms score from week 6 to week 12. This supports that notion that there is a build-up period at doses <1000 mg/day. It is entirely possible that a longer loading phase might be necessary in order to observe the changes in urological health. This is based on the potential antioxidant mechanism of action, in addition to the anti-inflammatory mechanism of action for GBTE.

Since polyphenols in tea have been detected in both animal and human prostate tissues, it is possible that in men with LUTS that the antioxidants in GBTE migrated to urological tissues, quenched site-specific free radicals, and asserted an anti-antioxidant effect; however, a longer loading period may have been necessary to exert its effectiveness on LUTS symptoms and CRP. Conversely, in the clinical trial with GBTE in generally healthy men which showed a dose-response serum anti-oxidant effect, there may have not been a site of concentrated inflammation or oxidative stress. This may have allowed the anti-oxidants in the tea blend to quench some harmful free radicals present throughout the body, with the remaining ones being detectable in the serum in a dose-response fashion.

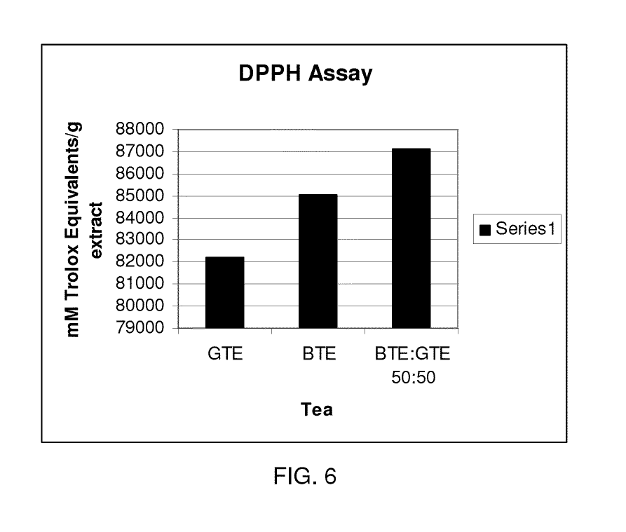

Finally, the literature indicates that this is the sole study evaluating the benefit of a combined green tea and black tea blend on antioxidant capacity. Data in FIG. 6 demonstrates the rationale for concluding that the observed effects are greater than those that would have been observed had green or black tea been administered alone. The DPPH assay was used to determine the level of antioxidant capacity by comparison to Trolox (a known antioxidant) in a black tea extract, green tea extract, or a black and green tea extract blend employing a UV/VIS spectrophotometric method based on reduction of a 2,2-Diphenyl-1-picryl-hydrazyl (DPPH) free radical. Blends of GTE and BTE showed higher antioxidant activity than either GTE or BTE alone (FIG. 6). A graphical comparison of the 50%/50% blend and GTE and BTE alone shows unexpectedly high antioxidant activity for the blend. These experimental observations are surprising because one skilled in the art would assume that by blending equal amounts of two different components one would achieve data that are approximately average of the two components, not produce data that are of significantly higher levels. The present blend of teas achieved significantly higher levels of antioxidant capacity than either individual tea products, resulting in a synergistic effect. Scientists have been studying antioxidants for 50 plus years and this has not been recorded especially with respect to teas.

### Conclusion

The present invention consists of a method for improving urological health, alleviating LUTS and BPH symptoms (e.g., post void residual volume, AUA Symptom Scores, nocturia, weak Stream, urinary frequency, urinary intermittency, incomplete emptying, inability to perform activities of daily living, and sexual desire and function), alleviating physiological risk factors (e.g., reducing inflammation [CRP], oxidative stress, blood pressure), by the administration of a therapeutically effective amount of a combination of water-extracted extracts of green and black tea. In addition, this green tea and black tea blend showed surprisingly greater capacity as an antioxidant than might be expected from either green tea or black tea alone.

### Example 2

The aim of this clinical study was to evaluate Kemin's GBTE in a human clinical study of healthy men in order to confirm its in vivo antioxidant capability and to monitor antioxidant levels, dose-response potential, and side effects/toxicities. In this study, GBTE was administered to healthy male subjects in a randomized, double-blind, placebo controlled study in order to test three doses of the study agent vs. placebo over a 4 week period. The study also measured safety, tolerability, compliance and other secondary outcomes. The hypothesis was that GBTE, a combination of green and black tea extracts, may be beneficial in increasing serum antioxidant levels in healthy male subjects.

### Patients and Methods

### Study Design

This was a prospectively designed, Institutional Review Board (IRB) approved (Quorum IRB, Seattle, Wash.), human clinical trial performed in accordance with Good Clinical Practices conducted at NIS Laboratories (Klamath Falls, Oreg.). Eligible participants included men in good health who were 25-70 years of age, had no course of newly prescribed medication within two weeks of the first study dose, were able to comply with the study requirements and were willing to provide written informed consent. Men were excluded if they had had known renal or hepatic insufficiency, scheduled elective surgery or other procedures requiring general anesthesia during the study, donated blood during the study or within the past month, participated in another research study involving an investigational product in the past month, were using any dietary supplement that might affect anti-oxidant status including vitamin E>4001U, vitamin C>500 mg, CoQ-10, lycopene, resveratrol, pycnogenol, or any formulation similarly named “Antioxidant formula”, or any other dietary supplement thought to affect antioxidant levels. Men who drank >1 cup of tea or >2 cups of coffee or energy drinks per day or had a history of any smoking within the past 3 months were excluded from the study (see Table 1 of Example 1). Subjects who met eligibility criteria were randomized to one of three study arms, 250 mg GBTE (administered as 2 capsules BID of 62.5 mg), 1000 mg GBTE (administered as 2 capsules BID of 250 mg) or placebo (also 2 capsules BID), for a total of 4 capsules per day for all groups. Subjects were instructed to maintain baseline consumption of medications and supplements as reported in the medical history, as well as not change diet and exercise habits throughout the intervention. Evaluable subjects were defined prospectively as those subjects completing the trial with a compliance of greater than 80% as measured by pill count.

The primary objective of this study was to determine the effects of a 4 week supplementation of GBTE on antioxidant status in healthy human male subjects. All measures were assessed at each time point, baseline (BL) and endpoint (week [wk] 4). A telephone call was performed at week two to remind subjects to comply with consumption of the study agent and to assess any adverse events. At BL and wk 4, subjects were asked to avoid food and drink except water for 10 hours prior to the blood draw. For the final visit, subjects were asked to take their last dose of the study agent the day before the scheduled visit (˜18 hours prior to blood draw). The following variables were evaluated for safety and tolerability of the product: adverse events, clinical laboratory tests (hematology and hepatic function) and vital signs (blood pressure, heart rate, oral temperature, and respiration rate). Adverse events were graded according to the severity (mild, moderate, severe, or life threatening) and relationship to the study medication (not related, unlikely related, possibly related, probably related, definitely related). Safety monitoring was continuously performed over the 4-week study duration and the participants were interviewed and examined by a study physician at the beginning, midpoint and end of the study. Adverse events were graded using National Cancer Institute, Common Terminology Criteria for Adverse Events, Version 3.0.

### Antioxidant Tests

An antioxidant panel of testing was performed that, Serum ferric iron reducing antioxidant power (FRAP) and Cellular antioxidant protection (CAP-e). Serum FRAP measures the reducing capability based on ferric iron. CAP-e is a test developed by NIS Laboratories (NIS Laboratories, Oregon) that assesses both bioavailability and ability to provide antioxidant protection at the cellular level and is useful for evaluation of serum changes in clinical studies.

### Study Agent

The study agent was an encapsulated dietary supplement, that contained 0, 62.5 mg or 250 mg of GBTE. Men were instructed to take 2 capsules twice a day with breakfast and lunch for a total dose of 0 mg (placebo), 250 mg GBTE or 1000 mg GBTE per day. The study agent was formulated under Good Manufacturing Practices, produced, encapsulated and packaged in 120 capsule quantities into light resistant plastic bottles. The product lots were tested for toxins including heavy metals, pesticides and excipients. The placebo agent contained an inert substance and was aesthetically matched to the study agent. Stability of the capsules was confirmed throughout the study period (data not shown).

### Statistical Analyses

Statistics are presented as mean±SEM for normally distributed variables (e.g., age). Primary outcomes were evaluated for both the intent to treat (ITT) and evaluable groups; however, all demographic and secondary analysis was done only on evaluable subjects. Demographic data for the 4 treatment groups were compared with ANOVA. For the primary outcome variables each group was compared to the placebo using a one-tailed t-test based on previous knowledge of the ability of tea to positively influence antioxidant status and all other outcome measures were evaluated using two-tailed t-tests. Statistical significance was defined at p≦0.05.

### Results

### Subjects

The 250 mg GBTE, 1000 mg GBTE, and placebo groups consisted of 12(10), 13(11), and 12(11) subjects (ITT [evaluable]), respectively. Treatment groups were well balanced in demographics (Table 3).

TABLE 3
Baseline Demographics (Evaluable Subjects)

| | Arm 1: | Arm 2: | Arm 3: | p- |
|---|---|---|---|---|
| Characteristic | 250 mg | 1000 mg | Placebo | value |
| N | 10 | 11 | 11 | n/a |
| Height (in) | 69.43 (2.27) | 70.20 (2.58) | 70.14 (3.05) | 0.955 |
| Weight (lb) | 198.05 (30.76) | 183.77 (29.27) | 212.68 (56.98) | 0.363 |
| BMI | 28.78 (3.15) | 28.45 (4.31) | 30.17 (7.08) | 0.218 |
| Age | 54.1 (2.93) | 49.8 (3.2) | 54.3 (4.2) | 0.572 |

Mean (SEM)

### FRAP

The mean percent change in FRAP from BL to wk 4 increased in a dose-dependent manner for the ITT group and the mean percent change from BL to wk 4 for the FRAP value increased versus placebo in the evaluable subjects consuming 250 and 1000 mg GBTE per day (Table 4). The mean percent change from baseline to wk 4 for FRAP values was 12.68±7.20%, 10.42±5.68%, and 19.44±12.61% in the evaluable 250 and 1000 mg GBTE groups respectively vs. placebo −6.32±5.11% (p=0.021 and p=0.036, for 250 and 1000 mg GBTE respectively).

TABLE 4
Antioxidant Measurements

| | Arm 1: | Arm 2: | Arm 3: |
|---|---|---|---|
| Characteristic | 250 mg | 1000 mg | Placebo |
| FRAP umol/L | | | |
| (SEM) | | | |
| ITT Day 0 | 3.54 (0.36) | 4.12 (0.3) | 4.14 (0.45) |
| ITT Day 28 | 3.907 (0.42) | 4.6 (0.37) | 3.77 (0.38)\hat{ } |
| Evaluable Day 0 | 5.933 (0.62) | 6.311 (0.93) | 6.29 (0.66) |
| Evaluable Day 28 | 6.807 (1.006)* | 7.71 (1.12)* | 5.72 (0.45) |
| CAP-e (SEM) | | | |
| ITT Day 0 | 30485 (1779) | 28919 (1648) | 28062 (1277) |
| ITT Day 28 | 29484 (5421) | 27823 (1742) | 29140 (1048) |
| Evaluable Day 0 | 31191 (2486) | 28919 (1648) | 29326 (1432) |
| Evaluable Day 28 | 29705 (2058) | 27823 (1742)* | 29933 (901.2) |

*<0.05 vs Placebo
\hat{ }0.05 vs Day 0

### CAP-e

The mean percent change from BL for CAP-e decreased (demonstrating improved antioxidant status) at wk 4 in the 1000 mg GBTE ITT dose group versus placebo (−4.068±1.784% [1000 mg GBTE], 3.162±3.504% [placebo], p=0.0339). There were no non-compliant individuals in the 1000 mg GBTE group; therefore the Evaluable data is the same as the ITT (Table 4).

### Vital Signs

There was a significant improvement in systolic blood pressure in the evaluable 250 mg GBTE group versus BL (125.90±3.05 vs. 120.10±2.52 mm Hg, day 0 vs. day 28, respectively, p=0.0478, Table 6). In the evaluable 1000 mg GBTE group there was a significant decrease in systolic (125.11±2.82 vs. 119.0±2.15 mm Hg) and a decreasing trend in diastolic (79.89±2.34 vs. 75.9±1.80 mm Hg) blood pressure at day 28 versus day 0 (p=0.017, p=0.0667 systolic, diastolic respectively).

TABLE 7
Evaluable Blood Pressure,

| | | | | P value | P value |
|---|---|---|---|---|---|
| | Arm 1: | Arm 2: | Arm 3: | 250 mg vs | 1000 mg vs |
| Category | 250 mg | 1000 mg | Placebo | Baseline | Baseline |
| SBP (mm HG) | | | | | |
| Day 0 | 125.90 (3.05) | 125.11 (2.82) | 126.75 (3.32) | 0.0478 | 0.017 |
| Day 28 | 120.10 (2.52)* | 119.0 (2.15)* | 125.83 (1.92) | | |
| DBP (mm HG) | | | | | |
| Day 0 | 76.60 (2.74) | 79.89 (2.34) | 78.18 (3.85) | 0.2354 | 0.0667 |
| Day 28 | 74.70 (2.58) | 75.89 (1.80) | 76.00 (2.75) | | |

p < 0.05

There was a significant difference in fasting serum blood glucose at Day 28 in the 250 mg GBTE group versus placebo (88.50±5.82 vs. 94.91±6.02 mg/dL, 250 mg GBTE and placebo, respectively), while a trend toward significance in the 1000 mg GBTE group versus placebo was observed (see Table 8 for glucose and other laboratory values).

TABLE 8
Laboratory Values

| | | | | p-value | p-value |
|---|---|---|---|---|---|
| | | | | 250 | 1000 |
| | | | | mg vs | mg vs |
| Cate- | Arm 1: | Arm 3: | Arm 4: | pla- | pla- |
| gory | 250 mg | 1000 mg | Placebo | cebo * | cebo * |
| Glucose | 90.20(8.28) | 92.18(8.55) | 93.27(7.35) | 0.379 | 0.752 |
| D 0 | | | | | |
| Glucose | 88.50(5.82) | 89.36(6.64) | 94.91(6.02) | 0.023 * | 0.054 |
| D 28 | | | | | |

* p < 0.05 ANOVA

### Safety and Tolerability

In subjects who completed the study, one reported adverse event deemed probably related to the study agent included a report of stomach upset and gas (1000 mg GBTE group). An additional reported adverse event of a laceration on the forearm (placebo) was reported. No subjects were discontinued because of any elevated liver function test, clinically significant abnormalities in any other laboratory assessment, or changes in any of the vital sign parameters. All subjects tolerated the subjects and no test-article related changes in clinical laboratories were identified.

### Discussion

The aim of this study was to analyze three different dosages of a novel green and black tea blend on serum antioxidant parameters, blood chemistries, vital signs, and to assess the safety and tolerability of study agent in healthy human male subjects. A panel of testing is beneficial in the evaluation of the antioxidant capabilities of an ingredient because antioxidant compounds may operate through multiple mechanisms of action.

This study showed that there were increased levels of serum FRAP in subjects taking any of the two doses of GBTE versus placebo at day 28, demonstrating the beneficial effect of the administration of GBTE on serum antioxidant levels. This is an important finding as it is crucial to understand the antioxidant capacity of a given dietary ingredient. The FRAP assay determines the antioxidant capacity of an ingredient by demonstrating reduction of ferric to ferrous ion at 593 nm based on the concept that a biologically active antioxidant is “any substance that, when present at low concentrations compared to those of an oxidizable substrate, significantly delays or prevents oxidation of that substrate” (Halliwell, B., and Gutteridge, J. M. C. (1995) Free Radicals Biol. 1321S. Med. 18, 125-126). A concurrent percentage change in CAP-e in the 1000 mg GBTE dose group also demonstrated GBTE's antioxidant capabilities as CAP-e measures the ability of cells to quench free radicals (Jensen G S, Ager D M, Redman K A, Mitzner M A, Benson K F, Schauss A G. Pain reduction and improvement in range of motion after daily consumption of an Acai (Euterpe oleracea Mart.) pulp-fortified polyphenolic-rich fruit and berry juice blend. J Med Food. 14 (7/8) 2011, 702-711). Given the approximate 18 hour duration between the last dose of the study agent and the wk 4 blood draw, a chronic antioxidant effect was achieved with GBTE supplementation. Taken together these results show that GBTE is a potent antioxidant and improved antioxidant status may be beneficial in promotion of health and possible prevention of a variety of diseases linked to chronic inflammation including cardiovascular disease, metabolic diseases such as diabetes mellitus and many cancers (Islam M A. Cardiovascular effects of green tea catechins: progress and promise. Recent Pat Cardiovasc Drug Discov. 2012 August; 7(2):88-99; Suliburska J, Bogdanski P, Szulinska M, Stepien M, Pupek-Musialik D, Jablecka A. Effects of green tea supplementation on elements, total antioxidants, lipids, and glucose values in the serum of obese patients. Biol Trace Elem Res. 2012 December; 149(3):315-22; Zheng P, Zheng H M, Deng X M, Zhang Y D. Green tea consumption and risk of esophageal cancer: a meta-analysis of epidemiologic studies. BMC Gastroenterol. 2012 Nov. 21; 12(1):165). Recent studies have also confirmed that both green and black teas are bioavailable and consumption favorably impact markers of oxidative stress including FRAP (Panza V S, Wazlawik E, Ricardo Schutz G, Comin L, Hecht K C, da Silva E L. Consumption of green tea favorably affects oxidative stress markers in weight-trained men. Nutrition. 2008 May; 24(5):433-42. doi: 10.1016/j.nut.2008.01.009. Epub 2008 March 12; Leenen R, Roodenburg A J, Tijburg L B, Wiseman S A. A single dose of tea with or without milk increases plasma antioxidant activity in humans. Eur J Clin Nutr. 2000 January; 54(1):87-92). Finally, in house ex vivo anti-oxidant data was confirmed in vivo in the current study at 1000 mg GBTE daily (data not shown).

This study also demonstrated significantly lower systolic blood pressure levels in both the 250 mg and 1000 mg GBTE groups at day 28 versus BL and a trend towards lower diastolic blood pressure in the 1000 mg GBTE group. A recent human study showed that green tea extract consumption (379 mg/daily of green tea extract [including 208 mg of EGCG]), significantly decreased both systolic and diastolic blood pressure by 4 points each after 3 months (145±10 at BL versus 141±8 at 3 months [p=0.004], and 88±4 versus 84±3 [p<0.001], systolic and diastolic, respectively). This study differed from the current evaluation in healthy subjects as this was a study of 56 subjects suffering from obesity and hypertension (Bogdanski P, Suliburska J, Szulinska M, Stepien M, Pupek-Musialik D, Jablecka A. Green tea extract reduces blood pressure, inflammatory biomarkers, and oxidative stress and improves parameters associated with insulin resistance in obese, hypertensive patients. Nutr Res. 2012 June; 32(6):421-7). Acute black tea administration has also been shown to increase pulse wave velocity, decrease Augmentation Index, and increase flow-mediated dilation in healthy subjects. (Jochmann N, Lorenz M, Krosigk A, Martus P, Bohm V, Baumann G, Stangl K, Stangl V. The efficacy of black tea in ameliorating endothelial function is equivalent to that of green tea. Br J. Nutr. 2008 April; 99(4):863-8. Epub 2007 Oct. 5; Vlachopoulos C, Alexopoulos N, Dima I, Aznaouridis K, Andreadou I, Stefanadis C. Acute effect of black and green tea on aortic stiffness and wave reflections. J Am Coll Nutr. 2006 June; 25(3):216-23).

In this study, subjects demonstrated significantly lower fasting serum blood glucose levels at 250 mg and 1000 mg GBTE groups versus placebo after administration of the study agent for only 4 weeks. These novel benefits in healthy individuals were detected sooner than a 3 month study by Bogdanski et al, which a demonstrated improved serum fasting blood glucose levels in obese, hypertensive subjects treated with green tea extract versus controls (5.5±0.4 vs 5.0±0.3 mmol/L [p=0.016], BL versus 3 months, respectively) (Bogdanski, et al., 2012).

Limitations of the current study include the short intervention period, however, the results obtained after only 4 wk of supplementation may indicate the potency of the study agent and warrants further long-term analysis. Strengths of the current study include its multi-dose, randomized, double-blind, placebo controlled design and evaluation of antioxidant status using several assays. This study demonstrated that 4 wk supplementation of GBTE improved serum antioxidant states, improved blood pressure and glucose and was safe and well-tolerated in healthy men.

### Conclusion

The administration of GBTE resulted in increases in serum antioxidant status, blood glucose levels, and systolic blood pressure at doses as low as 250 mg GBTE daily within 4 weeks. The administration of GBTE at up to 1000 mg daily was safe and well-tolerated.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. An antioxidant composition comprising a blend of an extract of green tea and an extract of black tea that has greater antioxidant activity than the same amount of either extract alone.

2. An antioxidant composition as claimed in claim 1, wherein the blend is between 30% and 70% green tea extract and between 70% and 30% black tea extract.

3. An antioxidant as claimed in claim 1 or claim 2 wherein the extracts are water extracts.

4. An antioxidant composition comprising a blend of an extract of green tea and an extract of black tea that has greater antioxidant activity than the sum of the antioxidant activity of the same amounts of the green tea and black tea extracts.

5. An antioxidant composition as claimed in claim 4, wherein the blend is between 30% and 70% green tea extract and between 70% and 30% black tea extract.

6. An antioxidant as claimed in claim 4 or claim 5 wherein the extracts are water extracts.

7. A method of improving urological health in a human, comprising administering an effective amount of the extract of claim 1.

8. A method of reducing the AUA Symptom Score in a human, comprising administering an effective amount of the extract of claim 1.

9. A method of reducing inflammation in a human, comprising administering an effective amount of the extract of claim 1.

10. A method of ameliorating lower urinary tract symptoms in a human, comprising administering an effective amount of the extract of claim 1.

11. A method of improving sexual desire in a human, comprising administering an effective amount of the extract of claim 1.

12. A method of improving quality of life through improved physical functioning, including but not limited to activities of daily living, in a human, comprising administering an effective amount of the extract of claim.

13. A method of ameliorating benign prostatic hyperplasia (BPH) in a human, comprising administering an effective amount of the extract of claim 1.

14. A method of ameliorating nocturia in a human, comprising administering an effective amount of the extract of claim 1.

15. A method of ameliorating weak stream in a human, comprising administering an effective amount of the extract of claim 1.

16. A method of ameliorating urinary frequency in a human, comprising administering an effective amount of the extract of claim 1.

17. A method of ameliorating urinary intermittency in a human, comprising administering an effective amount of the extract of claim 1.

18. A method of ameliorating incomplete emptying in a human, comprising administering an effective amount of the extract of claim 1.

19. A method of ameliorating oxidative stress in a human, comprising administering an effective amount of the extract of claim 1.

20. A method of reducing blood pressure in a human, comprising administering an effective amount of the extract of claim 1.

21. A method of reducing fasting blood glucose in a human, comprising administering an effective amount of the extract of claim 1.
